(19) United States
(12) Patent Application Publication
(10) Pub. No.: US 2025/0025082 A1
(43) Pub. Date: Jan. 23, 2025

(54) METHOD AND SYSTEM FOR TESTING COGNITION BY PROCESSING THE REACTION OF A SUBJECT TO STIMULI

(71) Applicants: Centre National De La Recherche Scientifique, Paris 16 (FR); It's Brain, Orsay (FR); Universite Paris-Saclay, Gif-sur-Yvette (FR)

(72) Inventors: Sandra Suarez, Bures-Sur-Yvette (FR); Bertrand Eynard, Bures-Sur-Yvette (FR); Sylvie Granon, Saint-Prest (FR)

(21) Appl. No.: 18/906,825

(22) Filed: Oct. 4, 2024

Related U.S. Application Data
(63) Continuation of application No. 17/597,676, filed on Jan. 18, 2022.

(30) Foreign Application Priority Data
Jul. 17, 2019 (FR) 1908081

(51) Int. Cl. A61B 5/16; A61B 5/00

(52) U.S. Cl. CPC A61B 5/162; A61B 5/163; A61B 5/748

(57) ABSTRACT

A system for processing the response of a human or animal subject to sensory stimuli comprises means for emitting these stimuli toward the subject, means for detecting responses of the subject to these stimuli, means for subjecting the subject to instructions to respond to these stimuli in response to an instruction previously taught to the subject, of increasing or decreasing complexity according to at least three difficulty levels, means for measuring a reaction time to simple stimuli corresponding to a first difficulty level and means for measuring a reaction time to stimuli linked to a choice on a categorization corresponding to a second difficulty level, and means for measuring a reaction time linked to a third difficulty level comprising a task in which the subject must simultaneously carry out two categorizations on the same stimulus.

### CROSS-REFERENCE TO RELATED APPLICATIONS

This application is a continuation of U.S. patent application Ser. No. 17/597,676, filed Jan. 18, 2022, which is a national phase entry under 35 U.S.C. § 371 of International Patent Application PCT/FR2020/051299, filed Jul. 17, 2020, designating the United States of America and published as International Patent Publication WO 2021/009470 A1 on Jan. 21, 2021, which claims the benefit under Article 8 of the Patent Cooperation Treaty to French Patent Application Serial No. FR1908081, filed Jul. 17, 2019.

### TECHNICAL FIELD

The present disclosure relates to a system for testing cognition by processing the reaction of a subject to stimuli. The present disclosure also relates to a method implemented in the system. The method and the system relate to the field of neuropsychology and neuroscience, and make it possible to test certain cognitive abilities involved in the reaction of a user to the difficulty, in order to aid the assessment of the cognitive function relating to the visual-motor decision-making capacities and including the attentive and executive functions, as well as the reaction to the difficulty.

### BACKGROUND

International Patent Application Publication No. WO2009137663 A1 relates to vision and cognition testing and/or learning under conditions of stress. In this case, the visual and cognitive abilities of a subject may be analyzed and/or trained by providing visual stimuli to a subject. More particularly, a subject may be evaluated and/or trained when they are under a condition of stress, in order to determine the effect of a condition of stress, such as physical stress or cognitive stress, on the visual and sensory capacities of the subject.

International Patent Application Publication No. WO2003015059 A1 discloses a system and a method, which make it possible to diagnose the emergence and monitor the development of an intellectual disability. The method may consist in performing one or more psychological tests and in teaching a subject the rules, which make it possible to respond to one or more tests without providing cultural signals, such as can be found in language-based learning techniques. Appropriate responses to the tests may be simulated during a learning phase preceding the test phase. An apparatus, a system, and a method for controlling a cognitive function can be implemented in a computerized system.

U.S. Pat. No. 6,632,174B1 relates to a method for testing and/or evaluating cognitive capacities. Depending on the results, the cognitive level can then be divided into distinct cognitive abilities, and one or more tasks can be created, each task being linked to each of the distinct cognitive abilities.

United States Patent Application Publication No. US20090287064 discloses a method implemented by a computer for cognitive tests, in particular, for ailments linked to dementia. The method is administered on a computer workstation in a web-based interactive format, and examines the cognitive capacities of the patient. The method makes it possible to give indications of dementia, including Alzheimer's disease and other cognitive issues. The method does not require administration by a qualified professional, but is rather administered in a Web browser, uses a server and a workstation via the Internet, or locally on the workstation.

U.S. Pat. No. 6,280,198B1 relates to a method implemented by a computer for administration and remote monitoring of cognitive tests on a person, using a computer network comprising separate computers. The computer-implemented method includes administering a set of basic cognitive tests to the person. The method further consists in repeatedly administering a set of cognitive tests, in obtaining a performance response from the person to the tests, and in downloading the test information via the computer network. A database may be constructed proceeding from the performance response of several people. The computer-implemented method comprises administration using at least two computers, at least one being local and the other being remote.

Canadian Patent CA 2683728 relates to the visual, cognitive and coordination abilities of a subject, which can be tested and/or trained by providing a variety of stimuli to a subject. The responses may be received from a subject, the appropriate nature of which may depend on the combination of stimuli provided to the subject. A touchscreen display device may be used both for providing stimuli and for receiving a subject's responses. Any type of output device and of input device for receiving the responses may be used. The information relating to the behavior, and other data relating to the performance of a subject, may be recorded. The rating may be based on the speed, the accuracy, and other aspects of the performance of a subject.

United States Patent Application Publication No. US 2005/0192513 A1 discloses a psychological test method for a subject, performed by presenting a test having output means such as a computer monitor and input means such as a keyboard, and by providing the subject with instructions playing a simulation of the test in order for the subject to learn how to perform the test from the simulation. The test is thus independent of the linguistic abilities of the subject or of the verbal instructions provided by a supervisor.

Document “The 3RT Test: Three reaction time tasks for IBM PC computers,” by Evelyn Lec Teng, published in Behavior Research Methods, Instruments, & Computers, 1 Jan. 1990 (1990 Jan. 1), pages 389-392, XP5569775, describes a test consisting in a simple task, a choice, and a conditional reaction time (ET). The three tasks involve visual stimuli, which are “comparable” but not strictly identical, since in the third task two symbols appear in place of a single one in the preceding two tasks (right-hand or left-hand arrow). The tasks require “identical” manual responses, where the subject presses on two buttons corresponding to the right and to the left, but they differ by the complexity of the cognitive processing required. The third task is a conditional reaction time task where the subject will respond to the right or to the left depending on the second symbol, which has appeared to the side; the response is conditional on the indication given by the second symbol, which appears beside the arrow. The non-verbal (visual) stimuli convey “generally known” meanings, i.e., arrows and “+, − or =” symbols. The responses may be made on a keyboard or on response buttons connected to the serial port of the computer.

The known methods do not disclose a more reliable measurement index of brain function, since they do not involve greater mobilization of the attention of the patient in response to strictly identical stimuli and also having a strictly identical motor response. A bias consequently exists on account of the variety (non-reliability) of the reaction time. Moreover, the prior art does not include filtering, which makes it possible to distinguish, among the patients' responses, those linked to the reaction to the difficulty from those linked to the attentive and executive cognitive state.

The prior art mainly uses simple or choice-based measurements of reaction time, or separate tests measuring specific functions with calculation of a “global” index. Subsequently, sets of tests are performed, it being understood that the majority of the tests are linked to the culture.

In order to assess the attentive and executive difficulties (relating to visual-motor decision-making capacities), it is necessary to evaluate the cognitive aspects and the reaction to the difficulty. This requires long series of tests. The majority of existing tests do not refer to recent neuroscience models, are linked to a given culture or language, and therefore cannot be used for all populations and have a learning effect (re-test effect), which prevents them from being re-used on the same person until several months or even years have passed.

The aim of the present disclosure is that of proposing a novel method for measuring visual-motor decision-making capacities (including attention, executive functions, inhibition, and reaction to difficulty), which is faster to administer, more international (not linked to the language or the culture of the subject), and is repeated at an interval of a few days or weeks (can be used for monitoring a follow-up), being in line with the latest developments in neuroscience.

### BRIEF SUMMARY

This object is achieved by a system for processing the responses of a human or animal subject to sensory stimuli, comprising:
means for emitting these stimuli toward a subject, and means for detecting responses of the subject to these stimuli,
means for subjecting the subject to instructions to respond to these stimuli, of increasing or decreasing complexity according to at least three difficulty levels,
means for measuring a reaction time to simple stimuli corresponding to a first difficulty level, and
means for measuring a reaction time to stimuli linked to a choice on a categorization corresponding to a second difficulty level, in response to an instruction previously taught to the subject.

According to the present disclosure, the processing system further comprises means for measuring a reaction time linked to a third difficulty level comprising a task in which the subject must carry out two simultaneous categorizations on the same stimulus.

The processing system according to the present disclosure may furthermore advantageously implement a stimuli library, which is common to all the difficulty levels.

It may further comprise a database for storing the responses detected, and means for extracting therefrom information on the cognitive and psychological functioning of the subject.

The database is advantageously intended for storing the historic performances of each subject. It may comprise images of animals according to different color gradients, and/or images of objects according to different color gradients.

The means for emitting a sensory stimulus toward a subject may comprise a screen or an audio headset or a sound emission device.

The means for detecting responses may be intended for receiving a pressure or gaze-fixing exercised continuously by the subject, and for detecting a relaxation of the pressure or the fixing, the relaxation being interpreted as a response to the stimulus.

The means for detecting responses, intended for permanently receiving a pressure or gaze-fixing practiced by the subject, and for detecting a relaxation of the pressure or fixing, may, for example, comprise a computer mouse, or a joystick, or a touchscreen, or a device for sensing the gaze direction, or any accessory that can sense the pressure or the gaze direction.

According to another aspect of the present disclosure, a method is proposed for processing the responses of a human or animal subject to sensory stimuli, implemented in the processing method according to the present disclosure, comprising a step of emitting the stimuli toward a subject, a step of detecting the responses of the subject to the stimuli, a step of processing the responses thus detected, the step of emitting stimuli being arranged so as to subject the subject to instructions for responding to the stimuli, of increasing or decreasing complexity according to at least three difficulty levels, the processing step comprising:
a step of measuring a reaction time to simple stimuli corresponding to a first difficulty level, and a step for measuring a reaction time to stimuli linked to a choice on a categorization corresponding to a second difficulty level, in response to an instruction previously taught to the subject.

According to the present disclosure, the processing step further comprises a step for measuring a reaction time linked to a third difficulty level comprising a task in which the subject must carry out two simultaneous categorizations on the same stimulus.

The set of emitted stimuli may be intended for distinguishing at least two choices, each choice referring to two categories, which are mutually exclusive.

The method according to the present disclosure can furthermore comprise processing of the reaction time corresponding to the first, second and third difficulty levels, comprising:
identifying errors caused by incorrect choices and/or early reactions and/or by no choice being made,
identifying errors caused by incorrect choices and/or early reactions and/or by reactions that are too slow,
access to the processing time of the difficulty associated with the categorization instructions of the subject, having removed, for each one, their simple reaction time corresponding to the visual detection and to the motive response, so as to deliver average executive reaction times,
mapping of the measurement results corresponding to the subject, on a plurality of axes comprising a first axis(S), which represents average executive reaction times, and a second axis (A), which represents the reaction of the subject to the difficulty of the stimuli.

“Average executive reaction time” means a reaction time linked to the categorization, i.e., not including the visual-motor response time.

The processing method according to the present disclosure may further comprise a step for placing the performance times of tasks of different difficultly levels into the same scale, in order to be able to compare them.

The detection of a response to a stimulus may comprise a detection of the relaxation of an action initiated previously and practiced continuously until the stimulus has arrived.

### BRIEF DESCRIPTION OF THE DRAWINGS

The present disclosure will be better understood with reference to the following figures:

FIG. 1 shows a practical example of a group of tests (A, B, C) implemented in the method according to the present disclosure, and the fact that each group of tests is preceded by a phase of learning the task;

FIG. 2 shows a particular embodiment of a response to a stimulus with attentive motor engagement (pressure engaging the test);

FIG. 3 shows the steps associated with part A of a test-measuring the reaction time of the subject;

FIG. 4 shows an embodiment of the database, without colors and with solid colors;

FIG. 5 shows the steps associated with part B of a test-measuring the reaction time associated with a category of choice;

FIG. 6 shows the steps associated with part C of a test-measuring the reaction time associated with two categories of choice;

FIG. 7 is an example of mapping of data of a set of measurement controls collected in the test method according to the present disclosure;

FIG. 8 shows an example of a table of correlations among the data collected;

FIG. 9 shows a method for graphical representation of the results of a control group and the axis of symmetry observed, and then the data represented, having a rotation of 45 degrees according to the axis of symmetry; and

FIG. 10 schematically shows the various measurements identified, errors, and times, and the mathematical processing steps carried out on the basis of the measurements.

### DETAILED DESCRIPTION

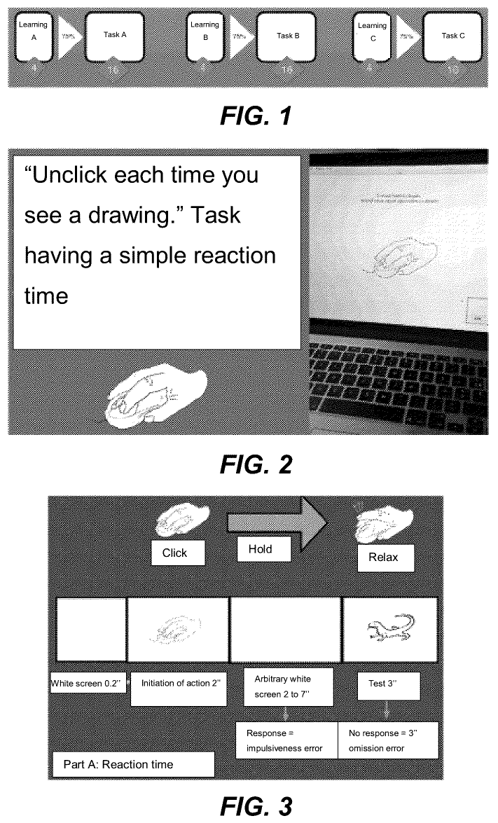

A practical embodiment of the testing method according to the present disclosure, comprising three parts A, B and C of increasing difficulty, each including 16 tests and preceded by a learning task, will now be described, with reference to FIGS. 1 to 6. The subject is placed in front of a computer screen and responds to the stimuli by means of a mouse connected to the computer. The number of images and their complexity are identical under all the conditions. The subject is invited to identify target differences under each condition.

This test makes it possible to show, on the same data, two axes, which are independent of the cognition of executive functions.

Using a mouse is one example, another object being able to implement the same function, such as a touchscreen, a keyboard, a joystick, a pedal, a stylus, and more generally any object sensing a pressure or an immobilization on a target followed by a change, for example, a gaze direction sensor.

For example, embodiments of the present disclosure may use various devices for testing a large range of motor skills, such as moving objects (change of location), and/or complex/continuous motor skills such as the positioning (acting on a command having discrete states) or control. The embodiments of the present disclosure can also measure movement time, regularity of the movements, complex hand-eye coordination, hand-hand coordination, foot-eye coordination.

The system may comprise software, as well as a combination of software and hardware: specially adapted joysticks, pedals, microphones, etc. It can furthermore be configured to function as an autonomous module located in the home of the user, in a medical center, an institution, a therapy center, or any other suitable location. It can furthermore comprise an interconnected system of one or more workstations. The system may comprise adaptable computer programs, which are accessible to the computers/workstations via CDs, a USB stick, the Internet, an intranet, or any other means allowing for information transport.

The measurements of the reaction time are in practice carried out using the internal computing means of the computer used for performing the test, but it is also possible to provide for the use of an external chronometry device, which would be connected to the computer.

The test method according to the present disclosure comprises, with reference to FIG. 2, elementary interrogation sequences consisting in pressing on a device, indicating the engagement of the subject's attention, a waiting time, the emission of a stimulus, and a response of the subject by maintenance or relaxation of the pressing. The stimuli may be visual (images) or auditory (sounds), and have properties in common (identical visual load, duration, frequency, identical sound volume, etc.). The stimuli each comply with or do not comply with criteria of the instruction (for example, gray or white, object or living, pitched low or high). The instruction is for the subject to relax the pressing if the stimulus meets all the criteria of the instruction, whether this be simple or complex.

The response is validated or otherwise, depending on whether the stimulus meets the criteria of the instruction. In the case of a good response, as in the case of a bad response, the time separating the emission of the stimulus and the response is measured.

In this embodiment, the test is performed by means of a computer and is based on a response or a “lifting of the finger from the mouse” after having verified the attentive engagement by moving the finger on the mouse. The action of the subject is measured so as to evaluate a speed of brain function, having removed the motor elements from the response.

Part A of the test according to the present disclosure is dedicated to measuring the simple reaction time of the subject. With reference to FIG. 3, a white screen is first displayed for 0.2 seconds, and then an invitation to perform a clicking action on the mouse appears for 2 seconds. A white screen is displayed for an arbitrary duration of between 2 and 7 seconds. If the subject responds during this sequence by relaxing their finger, this response will be interpreted as an impulsivity error. Following the white screen of arbitrary duration, a test image is displayed for 3 seconds. A failure to respond to this stimulus is interpreted as an omission error.

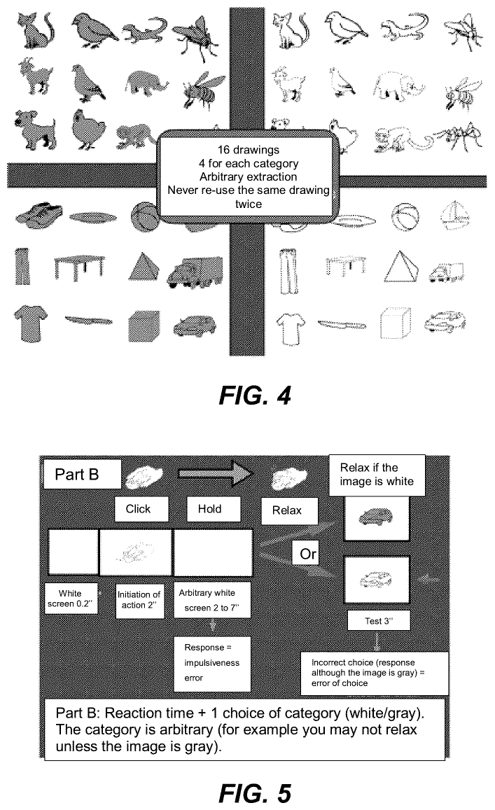

FIG. 4 shows an example of a test image displayed among the 16 images of a part A, B or C of the test, having 4 different images for each category. The images are extracted at random from a database, and no image is repeated.

In part B of the test, shown by FIG. 5, the objective is to measure the reaction time and a category choice (for example, white/gray). A choice criterion is randomized. This criterion is, for example, that the subject relax their clicking only if the image is gray. In this part B of the test according to the present disclosure, a reaction time to stimuli linked to a choice on a categorization corresponding to a second difficulty level, in response to an instruction previously taught to the subject, is measured.

The elementary sequence of the part B, inviting relaxation of the clicking if the image is white, is, for example, the successive displaying of a white screen for 0.2 seconds, an invitation to perform a click on the mouse for a period of 2 seconds, a random white screen for 2 to 7 seconds, followed either by a gray image or by a white image, for 3 seconds. The subject having made an incorrect choice, in this case having relaxed the pressure upon the appearance of the gray image, will be considered an error of choice.

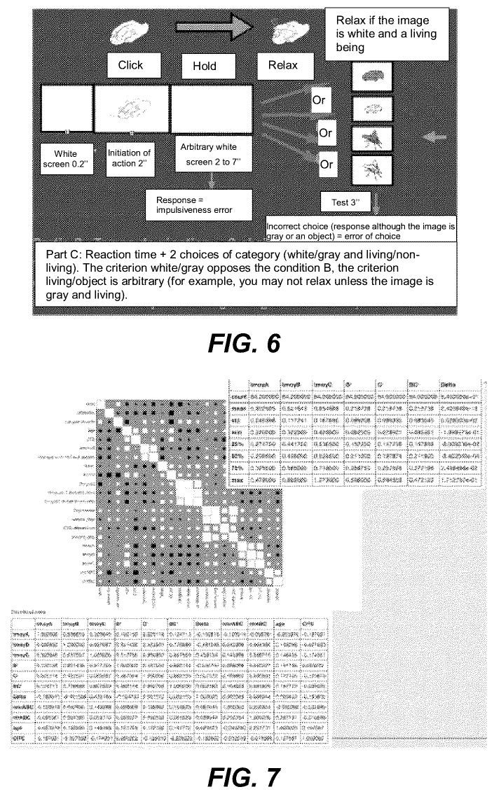

Part C of the test according to the present disclosure, shown by FIG. 6, is dedicated to measuring the reaction time and to the collection of two choices (white/gray, and living/non-living). The criterion for the white/gray choice is the opposite of condition B, while the criterion for the choice of living/non-living or object is randomized, for example, an invitation to relax the clicking only if the image is both gray and living. In this third part C of the test according to the present disclosure, the subject is subjected to a third difficulty level comprising a task in which the subject must carry out two simultaneous categorizations on the same stimulus.

Examples of processing of data collected by the test method according to the present disclosure will now be described, in particular, with reference to FIGS. 7 to 10.

The test method according to the present disclosure comprises a part that is intended to aid the understanding of the cognitive function of a subject by post-processing data of the subject with the aim of isolating two components of possible slowing in the subject. It is thus a case of understanding how the subject performs a decision-making process, by decomposing their cognition according to a set of underlying indicators of their cognitive function.

The data processing is carried out on normalized response times by removing the response time to the simplest experiment from the other response times, in order to eliminate the stimulus perception and action implementation parts of the response from the response time.

With reference to FIGS. 7 and 8, a correlation matrix between the characteristics/practices of 84 subjects considered normal and having acted as controls in the first calibration, and the processing of data collected for the three series of tests A, B and C, has been established.

On the basis of the data of normalized response times to experiments of increasing analytical difficulty, a two-factor analysis was performed, the factors being denoted S and A. These two factors have been demonstrated to be independent of one another. The first factor S is representative of an overall slowing of the subject from an average speed linked to the attention of the subject to the task put into a scale independent of the level of difficulty, and the second factor A is representative of excessive or reduced slowing in the event of an increase in the difficulty. These two factors can be used to characterize the cognitive function. These elements could then be used in neurological characterization and follow-up, in screening and follow-up of cognitive issues, in particular, in decision-making and attention or vigilance problems, in understanding of difficulties in the daily life of people, in order, in particular, to implement effective help strategies in daily life.

The cognitive function of a subject and the cognitive and psychological aspects linked to their decision-making are characterized, which makes it possible to statistically position their attentive and executive functions compared with the norms. In addition to these two factors, the analysis includes other factors based on an analysis of the rate and type of errors produced, and of the variance of the response times of each individual. This is because the aim of the test is to observe whether each of these factors deviates statistically from the norms.

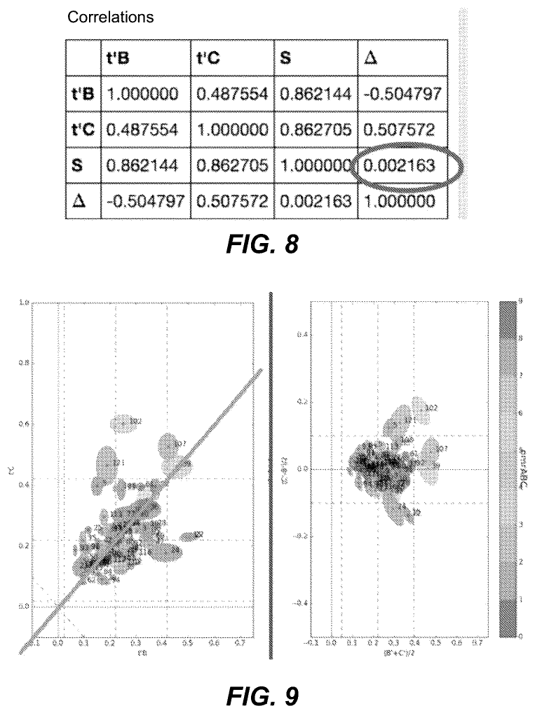

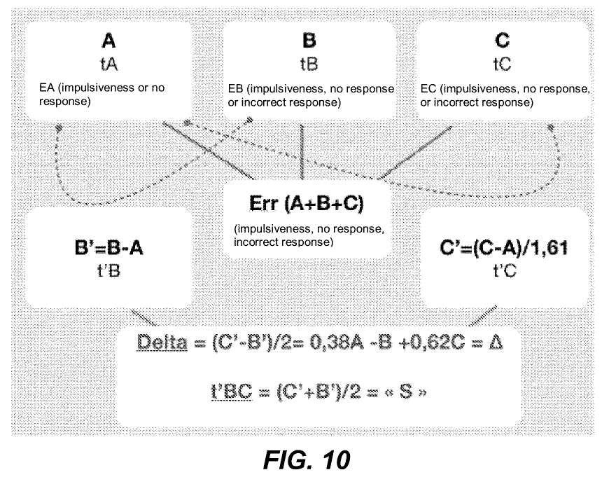

An example of the determination of a difficulty coefficient of the task will now be described, with reference to FIGS. 9 and 10. The principle is that of placing the performance times of tasks of different difficultly levels into the same scale, in order to be able to compare them.

For the reaction time A, it has been observed that this time has small fluctuations. It has been decided to subtract the time A, which is considered as a “motive (perception of the item and decision-making and performance of the motive response)” time base, since what is of interest is the “cerebral” extra time linked specifically to the analysis of the difficulty.

By means of this method, the times of different difficulty levels are put into the same scale, by means of a multiplication coefficient, in order to be able to compare the times. This change of scale is performed after subtracting the reaction time A. The coefficient 1.61 (non-limiting) has been established on the basis of the data of the 84 control subjects who served for the calibration, and the coefficient will be adjusted gradually as the calibration base increases, in particular, depending on the age and other parameters of the subject. The coefficient is calculated as the ratio of the average (over the control group) of C-A with respect to the average of B-A.

The reaction time A has been subtracted from two other conditions, which included additional difficulties:

$${B}^{’} = B - A and {C}^{’} = ( C - A ) / coefficient$$

The averages have thus been aligned (B′ and C′ have the same average over the control group). It is thus also observed that the differences between the two distributions are no longer visible. The standard deviations have become identical.

From a scientific viewpoint, this appears to mean that the remaining response time, the extra time required by the brain to analyze the difficulty, is a linear variable of the difficulty.

The two distributions were then compared and it was observed that it is not possible to statistically reject the hypothesis that they are identical.

In a chart 2D having B′=B−A on the X-axis and C′=(C−A)/coefficient on the Y-axis, as B′ and C′ have the same distributions, the graph of the distribution 2D is essentially symmetrical. Proceeding herefrom, the coordinates complying with the symmetry were sought. New coordinates S and Δ were thus defined, which are main component own directions.

Each time that the data are symmetrical, axes corresponding to this symmetry are selected. In the test method according to the present disclosure, a change of axis was thus performed. This is simply a different representation, without loss of information. By virtue of this symmetry, it is possible to view the half-sum and the half-difference, because it is known (as it is observed that B′ and C′ have the same standard deviations) that they are mathematically decorrelated.

For S, a reaction time (RT) of the subject is obtained, which is independent of the difficulty, and thus specific to the individual subject to the test. A representation of the attentive function, specific to each one, is obtained.

For Δ, in contrast, it is linked to the concept of increasing difficulty, and it represents the way in which a subject reacts to the increase in difficulty.
S is the average attention time, and corresponds to the average executive slowing of a person. In a scale made independent of the level of difficult of the tasks;
Δ is the variation of the response time linked to the increase in the difficulty of tasks, an index of reaction to the difficulty, including the reaction to the difficulty but also an index of the adaptation to the difficulty.

By way of example, shown by FIG. 10, the variables S and A may be calculated as follows:

$$Δ = ( {C}^{’} - {B}^{’} ) / 2 = 0 \text{.38} A - B + 0 \text{.62} C$$

$$S = {t}_{BC}^{’} = ( {C}^{’} + {B}^{’} ) / 2$$

where t′_{BC} represents an average executive reaction time of the subject undergoing the test, which is independent of the difficulty and thus specific to this subject.

Embodiments of the present disclosure can also be implemented on individual posts or on a plurality of workstations connected to a network in retirement homes and communities, or health clubs.

Embodiments of the present disclosure can also comprise a database, which can store the responses and optionally the historic performances of each user. The database may be local or remote, and may be accessible via the Internet, or may be entirely or partially available in the user's installations. In one embodiment, the database may be managed by an organization and may accumulate the histories and the responses of a number of individuals, thus allowing for the possible creation of a database of overall norms. The database may be used to compare the stored responses with the responses of the current user in order to facilitate the cross-validation of the user's test results with respect to the norms.

In this way, it may be possible to characterize the deviation of the current user with respect to the normal, and optionally use it to help the practitioner with determining the cognitive impairment linked to a condition such as a post-traumatic disorder (following a stroke or a head injury), Alzheimer's disease, Parkinson's disease (in particular, but not exclusively).

The tool for determining the cognitive function will thus constitute an element for aiding in the assessment of behavioral issues such as hyperactivity and attention deficit disorder (in particular, but not exclusively, using the set of tests relating to attention capacities), or learning difficulties such as dyslexia, dysgraphia or dyscalculia (mainly, but not exclusively, using fluidity measurement and naming tests). The uses of the database are not limited to these applications, and other applications thereof are included in the principle of the present disclosure.

It should be noted that embodiments of the present disclosure may be able to be exploited proceeding from a mobile storage device, such as a CD or a USB stick, or may be accessible from a central computer or via public communications networks such as the Internet. Moreover, embodiments of the present disclosure can be implemented, in practice, by a single person or an organization, and the database may be centralized, local and/or held by an internal or external organization.

Of course, the present disclosure is not limited to the embodiments described above, and a number of developments can be made to the embodiments, without departing from the scope of the invention as defined by the claims. Moreover, the various features, types, variants, and embodiments of the present disclosure may be associated with one another, in accordance with various combinations, insofar as they are not mutually incompatible or exclusive.

What is claimed is:

1. A system for processing responses of a human or animal subject to sensory stimuli, comprising:
means for emitting these stimuli toward a subject, and means for detecting responses of the subject to these stimuli,
means for subjecting the subject to instructions to respond to these stimuli, of increasing or decreasing complexity according to at least three difficulty levels,
means for measuring a reaction time to simple stimuli corresponding to a first difficulty level,
means for measuring a reaction time to stimuli linked to a choice on a categorization corresponding to a second difficulty level, in response to an instruction previously taught to the subject,
means for measuring a reaction time linked to a third difficulty level comprising a task in which the subject must carry out two simultaneous categorizations on the same stimulus,
processing means for processing the reaction times measured in response to the stimuli,
wherein the processing means are arranged so as to:
determine normalized reaction times, by subtraction of the response time to simple stimuli from the response time to stimuli linked to a choice and to stimuli linked to two simultaneous categorizations,
analyze the normalized reaction times according to two independent factors (S, Δ), which are representative, respectively, (i) of average executive reaction times and (ii) of the reaction of the subject to the difficulty of the stimuli,
map the measurement results corresponding to the subject, on a plurality of axis comprising a first axis (S), which represents average executive reaction times, and a second axis (Δ), which represents the reaction of the subject to the difficulty of the stimuli, and
place the normalized reaction times of tasks of different difficulty levels into a same scale, in order to be able to compare them.

2. The system of claim 1, wherein the system is configured to implement a stimuli library common to all the difficulty levels.

3. The system of claim 1, further comprising a database for storing the responses detected, and means for extracting therefrom information on the cognitive and psychological functioning of the subject.

4. The system of claim 3, wherein the database stores historic performances of each subject.

5. The system of claim 4, wherein the database comprises images of animals according to different color gradients.

6. The system of claim 3, wherein the database further comprises images of objects according to different color gradients.

7. The system of claim 1, wherein the means for emitting a sensory stimulus toward a subject comprise a screen, an audio headset, or a sound emission device.

8. The system of claim 1, wherein the means for detecting responses are intended for receiving a pressure or gaze-fixing exercised continuously by the subject, and for detecting a relaxation of the pressure or the fixing, the relaxation being interpreted as a response to the stimulus.

9. The system of claim 8, wherein the means for detecting responses, intended for permanently receiving a pressure or gaze-fixing practiced by the subject, and for detecting a relaxation of the pressure or fixing, comprise a computer mouse, or a joystick, or a touchscreen, or a device for sensing the gaze direction, or any accessory that can sense the pressure or the gaze direction.

10. A method for processing the responses of a human or animal subject to sensory stimuli, the method comprising:
a step of emitting the stimuli toward a subject, the step of emitting the stimuli including subjecting the subject to instructions for responding to the stimuli, of increasing or decreasing complexity according to at least three difficulty levels,
a step of measuring a response time of the subject to the stimuli, comprising:
a step for measuring a reaction time to simple stimuli corresponding to a first difficulty level,
a step for measuring a reaction time to stimuli linked to a choice on a categorization corresponding to a second difficulty level, in response to an instruction previously taught to the subject,
a step for measuring a reaction time linked to a third difficulty level comprising a task in which the subject must carry out two simultaneous categorizations on the same stimulus,
a processing step for processing the response times thus measured,
wherein the processing step comprises:
a step for generating normalized response times, by subtraction of the response time to simple stimuli from the response time to stimuli linked to a choice and to stimuli linked to two simultaneous categorizations, and
a step for analyzing the normalized response times according to two independent factors (S, Δ), which are representative, respectively, (i) of average executive reaction times and (ii) of the reaction of the subject to the difficulty of the stimuli,
wherein the set of emitted stimuli is intended for distinguishing at least two choices, each choice referring to two categories, which are mutually exclusive,
wherein the processing of the reaction times corresponding to the first, second and third difficulty levels comprises:
identifying errors caused by incorrect choices and/or early reactions and/or by no choice being made,
identifying errors caused by incorrect choices and/or early reactions and/or by reactions that are too slow,
access to the processing time of the difficulty associated with the categorization instructions of the subject, having removed, for each one, their simple reaction time corresponding to the visual detection and to the motive response, so as to deliver average executive reaction times,
mapping of the measurement results corresponding to the subject, on a plurality of axes comprising a first axis(S), which represents average executive reaction times, and a second axis (A), which represents the reaction of the subject to the difficulty of the stimuli, and
placing the performance times of tasks of different difficulty levels into a same scale, in order to be able to compare them.

11. The method of claim 10, wherein the stimuli comprise images.

12. The method of claim 10, wherein the stimuli comprise sounds.

13. The method of claim 10, wherein the detection of a response to a stimulus comprises a detection of the relaxation of an action initiated previously and practiced continuously until the stimulus has arrived.
